(19) United States
(12) Patent Application Publication
(10) Pub. No.: US 2004/0076646 A1
(43) Pub. Date: Apr. 22, 2004

(54) Haptenizing cancer cell components

(76) Inventors: Michael J. Caplan, Woodbridge, CT (US); H. Kim Bottomly, New Haven, CT (US)

Correspondence Address: Choate, Hall & Stewart, Exchange Place, 53 State Street, Boston, MA, 02109, US

(21) Appl. No.: 10/197,376

(22) Filed: Jul. 17, 2002

Related U.S. Application Data
(60) Provisional application No. 60/306,228, filed on Jul. 18, 2001.

(51) Int. Cl. A61K 39/00; C12N 5/08

(52) U.S. Cl. 424/277.1; 435/366

(57) ABSTRACT

The present invention provides techniques and reagents that induce an immune response against tumor cells. According to the invention, tumor cell components are contacted with a sensitizing agent, preferably so that one or more tumor cell components become haptenized with a sensitizing agent, and the resulting combination is administered to a patient suffering from a tumor, so that an anti-tumor immune response is mounted. In some embodiments of the invention, sensitizing agent/tumor cell component compositions are administered directly to a patient; in other embodiments they are administered by means of an antigen presenting cell such as a dendritic cell.

### PRIORITY INFORMATION

This application claims priority to U.S. Provisional Application No. 60/306,228, filed Jul. 18, 2001, which is incorporated herein by reference in its entirety.

### BACKGROUND OF THE INVENTION

Cancer cells utilize a wide variety of mechanisms to avoid host immune systems. For example, some researchers have proposed that self-tolerance mechanisms, relying on the similarities between tumor cells and non-tumor cells, allow cancer cells to avoid potent immune system attacks (see review by Sznol, PPO Updates 13:1, 1999). Particular self-tolerance mechanisms that have been proposed to contribute include the thymic deletion of T cells having high affinity for self antigens that is thought to occur during T cell development, the peripheral tolerance that can be induced if self-antigens on tumor cells are presented to T cells in the absence of co-stimulatory signals that are required for T cell activation (see, for example, Van Belle et al., J. Invest. Dermatol. 102:a553, 1994; Danfeld et al., Int. J. Cancer 62:259, 1995), and the sequestration of tumor-specific antigens within the tumor mass, where circulating T cells may not encounter them with sufficient frequency to induce an aggressive immune response.

Other researchers have explained the minimal immune responses mounted against tumor cells by invoking changes that occur in some tumor cells that could disrupt the ability of those cells to present tumor-associated antigens to the host immune system. For example, some tumor cells carry mutations or deletions of major histocompatibility complex (MHC) genes that encode proteins responsible for displaying tumor-associated antigen fragments on the surfaces of tumor cells; other tumor cells have defects in the antigen processing and/or transport systems that are required to achieve antigen fragment display (see, for example, Ferrone et al., Immunol. Today 16:487, 1995; Hicklin et al., J. Clin. Invest. D'Urso et al., J. Clin. Invest. 87:284, 1991).

Also, many tumor cells secrete cytokines and/or growth factors that may influence the activity of local immune system cells. For example, some tumors secrete IL-10 and vascular endothelial growth factor, which can interfere with antigen presenting cell maturation and therefore with antigen presentation in the vicinity of a tumor (see, for example, Bennicelli et al., Exp. Dermatol 2:186, 1993; Chen et al., Int. J. Cancer 56:755, 1994). Some tumors even produce specific proteins (e.g., FasL) that directly induce apoptosis in some T cells (Abbas et al., Cell 84:655, 1996; O'Connell et al., J. Exp. Med. 184:1075, 1996; Hahne et al., Science 274:1363, 1996; Keanne et al., Cancer Res. 56:4791, 1996).

A variety of approaches are now being developed to overcome or counteract the ability of tumor cells to avoid immune system attack. Both non-specific approaches, involving generalized stimulation of the immune system, for example, through administration of adjuvants (e.g., Bacille Calmette-Guerin (BCG), DETOX, QS-21, etc) and specific approaches, intending to target particular tumor cells, are being pursued. Efforts have been made to induce immune reactions to a patient's own tumor cells, or to allogenic cell lines, by administering cells or cell components together with one or more adjuvants or cytokines. In other work, particular tumor-associated antigens have been identified and efforts have been made to increase a host immune system's responsiveness to those antigens. Other approaches involve manipulating tumor cells to express certain selected cytokines (see, for example, Fearon et al., Cell 60:197, 1990; Becker et al., J. Exp. Med. 183:2361, 1996; Gilboa et al., semin. Oncol. 23:101, 1996; Parmiani et al., Adv. Pharmacol. 40:359, 1997), and modification of effector cells such as APCs so as to encourage presentation of tumor-associated antigens (see, for example, Ashley et al., J. Exp. Med. 186;1177, 1997; Schuler et al., J. Exp. Med. 186:1123, 1997).

Despite all of these efforts, there remains a need for an improved system for inducing an immune reaction against tumor cells. The present invention provides haptenized tumor cell components that induce a tumor-cell specific immune reaction.

### SUMMARY OF THE INVENTION

The present invention provides compositions and methods that stimulate the immune system of a host to mount an attack on tumor cells and/or antigens. In certain embodiments of the invention, tumor cells or cell components are combined with a preparation including one or more sensitizing agents, and subsequently are administered to a host containing one or more tumors. In certain preferred embodiments, one or more sensitizing agents may become covalently linked to one or more tumor cell components, so that the components are said to be “haptenized”.

In certain preferred embodiments of the invention, tumor cells are isolated from a host, are exposed to sensitizing agent, and are returned to the host. In particularly preferred embodiments, one or more tumor-associated compounds is prepared from the tumor cells prior or subsequent to exposure to the sensitizing agent. In other preferred embodiments of the invention, tumor cell components are obtained from tumor cells that did not originate in the host to which the inventive tumor cell component/sensitizing agent composition is to be administered.

In some embodiments, the present invention provides a composition that includes a tumor cell component and a hapten linked to the tumor cell component to form a haptenized tumor cell component. The hapten is characterized in that it maintains the ability to stimulate, support, or enhance an immune reaction in a host when linked to the tumor cell component. Particularly, the hapten is a compound naturally produced by poison ivy, poison oak, or poison sumac plants, which typically include urushiols. The tumor cell component is characterized in that it includes at least one intact cancer cell or tumor associated peptide. Where the tumor cell component is a tumor associated peptide, the hapten may be linked to the tumor-associated peptide as a hapten-peptide conjugate, which conjugate is present in an MHC cleft on a surface of the antigen presenting cell.

The present invention also provides a method of treating cancer by administering to a subject suffering from cancer a composition including an effective amount of a tumor cell component linked to a hapten to form a haptenized tumor cell component. In certain preferred embodiments, an antigen presenting cell is also be administered to a subject in combination with a haptenized tumor cell component. In related embodiments, the antigen presenting cell is loaded in vitro with the haptenized tumor cell component and subsequently administered to a subject in vivo for treatment of cancer. This provides a way to target the hapentized tumor cell component in vivo to the immune system of the subject.

The present invention provides a second composition that includes an antigen presenting cell, wherein the antigen presenting cell presents a sensitizing agent, such as a compound that is naturally produced by poison ivy, poison oak, or poison sumac plants, which contain urushiols. The antigen presenting cell may alternatively be loaded with the extract of poison ivy, poison sumac, or poison oak, or loaded directly with a urushiol. As described above, the antigen presenting cell may be any antigen presenting cell, such as a dendritic cell or a macrophage, and the like. In other preferred embodiments, the antigen presenting cell is loaded with a haptenized tumor cell component, which is administered to a subject in vivo, e.g., for treatment of cancer.

In a related embodiment, the present invention provides a method of stimulating an immune response in an individual by administering to a subject suffering from cancer a composition including and effective amount of a antigen presenting cell, wherein the antigen presenting cell presents a sensitizing agent that is a compound naturally produced by poison ivy, poison oak, or poison sumac plants, e.g., a urushiol or an extract containing a urushiol, as described above. In certain preferred embodiments, the sensitizing agent is present in an MHC cleft on the surface of the antigen presenting cell.

It will be appreciated that the haptenized tumor cell components of the invention may be encapsulated. Certain encapsulation devices and other available reagents for encapsulating biological reagents allow the targeting of the haptenized tumor cell components to antigen presenting cells. Another aspect of the invention involves administrating the haptenized tumor cell component with any known adjuvant. It will further be appreciated that the haptenized tumor cell components may be administered with other therapeutic agents, such as chemotherapeutic agents or anti-angiogenesis factors.

### Definitions

Adjuvant, as used herein, include agents that stimulate antigen presenting cells to enhance T cell response. Exemplary adjuvants include BCG, alum, Freund's, DETOX, QS-21, CpGs, Listeria, and the like.

Hapten, as used herein, refers to a sensitizing agent that is further characterized in that it becomes covalently linked to another compound, thereby “haptenizing” that compound. The hapten/compound complex preferably maintains the hapten's ability to induce, stimulate, support, or enhance an immune reaction in a host. Preferred haptens include, but are not limited to, compounds that are naturally produced by poison ivy, poison oak, or poison sumac plants. Particularly preferred haptens include urushiols.

Sensitizing agent, as that term is used herein, refers to a compound that is characterized by an ability to induce, stimulate, support, or enhance an immune reaction in a host. That is, a sensitizing agent is a compound that, when combined with one or more tumor cell components according to the present invention and delivered to an individual, attracts components of the host's immune system to mount an attack against the tumor cell components, and preferably against other tumor cells. Preferred sensitizing agents include compounds that are naturally produced by poison ivy, poison oak, or poison sumac plants. Particularly preferred sensitizing agents include urushiols.

Tumor-associated, as that term is used herein, refers to an entity that is preferentially associated with tumor cells, as compared with non-tumor cells of the same or a comparable lineage. As those of ordinary skill in the art will readily appreciate, it is generally preferable to compare tumor and non-tumor cells that are as closely related as possible when determining whether a particular entity is preferentially associated with tumor cells. Furthermore, it is often desirable to compare the presence or level of the entity of interest in a variety of different tumor/non-tumor cell pairs in order to firmly establish tumor association. Nonetheless, for the purposes of the present invention, preferential association in a single tumor/non-tumor cell pair may be sufficient to establish a given entity as being tumor associated. Preferably, an entity is present in, on, or around (depending on the nature of the entity) tumor cells at a level at least 1.5 fold, more preferably at least about 2, 5, 7, 10, 20, 30, 40, 50, 100, 500, 750, 1000, 10,000, etc. higher than the level at which is it present in, or, or around matched non-tumor cells.

Tumor specific, refers to a tumor-associated entity whose preference for tumor cells is sufficiently great (e.g., at least about 10, 20, 30, 40, 50, 100, 500, 750, 1000, 10,000, etc. fold) to justify labeling the entity tumor specific.

Tumor cell refers to a malignant or pre-malignant cell of any type of cancer. Those of ordinary skill in the art will appreciate that a “pre-malignant” cell may be a cell having abnormalities suggestive of cancer (e.g., a cervical cell having displastic changes, a skin cell having displastic nevi, etc.) but not necessarily fully cancerous.

Tumor cell component, as that term is used herein, is any compound or entity that is found in or is produced by a tumor cell. An intact tumor cell, or a collection of tumor cells, also qualifies as a tumor cell component according to the present invention. Preferred tumor cell components include tumor cells, purified components of tumor cells (e.g., a preparation of surface membrane proteins and/or tumor-cell fractions), and tumor-specific antigens (e.g., prostrate specific antigen (PSA)).

### DESCRIPTION OF THE DRAWING

FIG. 1 presents a urushiol structure.

### DESCRIPTION OF CERTAIN PREFERRED EMBODIMENTS

Sensitizing Agents and Haptens

Preferred sensitizing agents and haptens for use in the practice of the present invention include compounds that are naturally produced by poison ivy, poison oak, or poison sumac plants. Particularly preferred compounds are urushiols.

Poison ivy, poison oak, and poison sumac are classified into either the Rhus or the Toxicodendron genus within the family Anacardiaceae (for information about plant classification, see, for example, Cronquist, An Integrated System of Classification of Flowering Plants, Columbia University Press, New York, 1981; Reveal, Indices Nominum Supragenericorim Plantarum Vascularium, http://matrix.nal.usda.gov: 8080/star/supragenericname.html; Takahtajan, Diversity and Classification of Flowering Plants, Columbia University Press, New York, 1997, each of which is incorporated herein by reference). These plants, and other members of the Anacardiaceae family, produce urushiols.

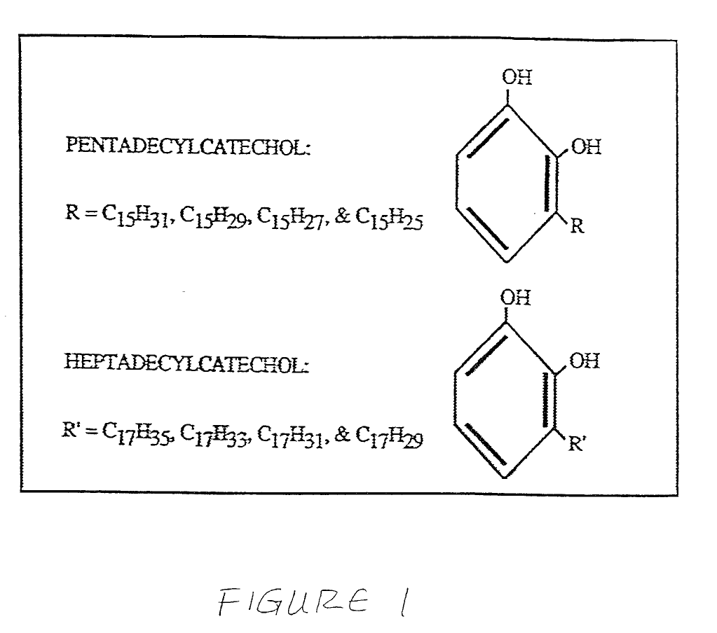

Urushiols are phenolic compounds with long (15-17 carbons) hydrocarbon side chains (see, for example, FIG. 1). The side chain may be saturated or unsaturated with 103 double bonds (see, for example, Dawson, Recent Chemical Progress 15:39, 1954; Dawson, Transcac. NY Acad. Sci. 18:427, 1956). Urushiol produced by poison oak plants is comprised primarily of compounds with 17-carbon side chains; that produced by poison ivy and poison sumac plants is comprised primarily of compounds with 15-carbon side chains.

As is well known, contact with poison ivy, poison oak, or poison sumac plants can cause severe contact dermatitis. It is thought that urushiols contribute to this reaction; other plants (e.g., members of the cashew genus Anacardium or the mango genus Magnifera) that also produce urushiols can have the same effect. Indeed, urushiols are supremely potent inducers of immune reactions. In some individuals, molecular traces of urushiol (≦2 μg) are sufficient to induce the rash response; 80-90% of adult Americans respond to ≦50 μg of purified material (Epstein et al., Arch. Dermatol. 109:356, 1974).

Without wishing to be bound by any particular theory, we note that urushiols may readily be oxidized to a more reactive quinone species that is capable of binding to proteins and other factors (see, for example, Dunn et al., Cell Immunol 74(2):220-33, December 1982). It is thought that urushiols from poison plants are converted to the quinone form when they penetrate the epidermal layer of skin, and that it may be the quinone form that actually elicits the immune response that results in contact dermatitis. For the purposes of the present invention, it is generally desirable to utilize a form of the compound that effectively elicits an immune response. Furthermore, preferred embodiments of the invention require haptenization of the sensitizing agent with tumor cell components, so that a more reactive compound will often be formed. Accordingly, quinone forms of urushiols are explicitly included within the scope of the invention and are considered particularly preferred for use in inventive sensitizing agents. Other urushiol derivatives with high reactivity and immunogenicity are similarly preferred. For example, some have reported that radical species, rather than or in addition to quinone species, may participate in or be responsible for urushiol immunologic activity (see, for example, Schmidt et al., Arch Dermatol Res 282(1):56-64, 1990). Thus, as used herein, the term “urushiol” is intended to refer both to the compounds as they are found in plants and the quinone derivatives, or other derivatives that are similarly capable of forming associations with proteins or other components of tumor cells (see, for example, Stampf et al., J Invest Dermatol 86(5):535-8, May 1986). Furthermore, it will be appreciated that sensitizing agents for use in the practice of the present invention are not limited to preparations containing urushiols. To the extent that non-urushiol plant compounds, or wholly unrelated compounds, achieve the inventive goals with respect to immunotherapy of tumors, they are encompassed within the spirit and scope of the invention.

Sensitizing agents for use in accordance with the present invention may be prepared according to any known technique. Sensitizing agents may be provided as crude extracts or mixtures, or alternatively may represent purified preparations of one or more compounds. Sensitizing agents may be isolated from natural sources, for example using known extraction and/or purification techniques, or may be synthesized in vitro using available synthesis technologies.

In general, extraction and purification procedures typically involve the separation of desired components of a plant or animal tissue from components that are considered undesirable, or even inactive, in the relevant context. A wide variety of such procedures are known, and standards have been established for a number of extracts (see, for example, Nairn in Remington's Pharmaceutical Sciences, 18th ed., 1990; United States Pharmacopeia—National Formulary (e.g., U.S. Pat. No. XXI/NF XVII, U.S. Pharmacopeial Convention, Inc., 1990), each of which is incorporated herein by reference). Preparation of extracts typically involves placing solid ingredients (e.g., plant or animal tissue) in a closed container with a solvent and allowing the mixture to stand, with or without agitation, for a variable period of time to allow soluble matter to dissolve. Alternatively or additionally, solvent can be percolated through a mass, optionally compacted, of raw material, and the percolate collected. Subsequently, the material mass can be compressed to express any additional liquid, that can be added to the percolate.

Following collection of an extract liquid, desired components may be concentrated, for example, by evaporation, or may be fractionated, for example by chromatography. Generally, the progress of desired components through steps of the extraction/purification procedure is followed through the use of biological (e.g., functional or immunological), chemical (e.g., reactive), or and/or physical (e.g., spectroscopic [see, for example, Workman (ed), Applied Spectroscopy. A Compact Reference for Practitioners, Academic Press, New York, 1998, incorporated herein by reference], chromatographic [see, for example, Fried et al. (eds), Practical Thin-Layer Chromatography: A Multidisciplinary Approach, CRC Press, Boca Raton, Fla., 1996; McMaster et al., GC/MS: A Practical User's Guide, Wiley & Sons, New York, 1998, each of which is incorporated herein by reference], etc.) assays.

As mentioned above, certain preferred inventive sensitizing agents include compounds produced by poison ivy, poison oak, poison sumac, or various other plants. A variety of different preparations of poison ivy, poison oak, and poison sumac components are known in the art (see, for example, Hasegawa et al., J. Chem. Soc. 72:223, 1951; Beretz et al., in Plant Flavonoids in Biology and Medicine: Biochemical, Pharmacological and Structure-Activity Relationships, A. R. Liss, New York, pp. 179-187, 1988; Kappus et al., Pharmacol 300:179, 1977; Baumann et al., Prostagladins 20:627, 1980; Yoshimoto et al., Biochem, Biophys,. Res. Commun. 116:612, 1983; Loggia et al. in Plant Flavenoids in Biology and Medicine, A. R. Liss, New York, pp. 481-484, 1986; Hong et al., Arch Pharm Res 22(6):638-41, December1999; Oelrichs et al. Nat Toxins 5(3):96-8, 1997; Kalisch et al., J Clin Invest 82(3):825-32, September 1988; Elsohly et al., J Pharm Sci 69(5):587-9, May 1980; Dupuid et al., Br J Dermatol 101(6):617-24, December 1979; Corbett et al., J Pharm Sci 64(10):1715-8, October 1975; Epstein et al., Arch Dermatol 109(3):356-60, March 1974; Na et al., U.S. Pat. No. 6,099,845, issued August 8, 2000, each of which is incorporated herein by reference).

Furthermore, syntheses have been developed for various urushiols and urushiol derivatives (see, for example, Byck et al., J Org Chem 32(4):1084-8, April 1967; Kurtz et al., J Med Chem 14(8):733-7, August 1971; Kurtz et al., J Org Chem 37(17):2767-8, August 25 1972; Lepoittevin et al., J Med Chem 29(2):287-91, February 1986; ElSohly et al., J Med Chem 29(5):606-11, May 1986; Stampf et al., J Invest Dermatol 86(5):535-8, May 1986; Dunn et al., Cell Immunol 97(1):189-96, January 1986, each of which is incorporated herein by reference).

Tumor Cell Components

Those of ordinary skill in the art will appreciate that any tumor cell component may be utilized in the practice of the present invention. Tumor cell components may originate from tumors of the following non-limiting types: melanoma, breast, lung, colon, kidney, prostate, and the like. In addition, mammals particularly humans, having metastatic cancer may be treated with the compositions and methods of the present invention.

In certain embodiments of the invention, intact tumor cells are employed. In other preferred embodiments, tumor cells are isolated from the patients to be treated. A tumor cell may be a malignant or pre-malignant cell of any type. According to the present invention, pre-malignant refers to any abnormal cell suggestive of a cancer cell, which is not yet a cancer cell; such as, but not limited to, displastic changes in cervical cells that ultimately lead to cervical cancer, and displastic nevi that are abnormal skin cells that lead to melanoma. Preferably, the tumor cells originate from the type of cancer that is to be treated. Such tumor cells may be, but are not limited to, autologous and allogenic cells dissociated from biopsy specimens or tissue culture, as well as stem cells and extracts from these sources. Tumor cell extracts may be extracts of whole cells, cell membranes. Alternatively, a tumor cell extract may be a peptide isolated from the cancer cell or membrane or haptenized cancer cell or membrane. Methods of preparing such tumor cells, tumor cell extracts, membranes, and peptides are described in WO 96/40173, incorporated herein by reference in its entirety.

For example, the cancer cell membranes of the present invention may be all or part of a membrane isolated from a cancer cell or a haptenized cancer cell. Surgical and other techniques for obtaining such tumor cells, for example, biopsy techniques are known in the art (see, for example, WO 96/40173, incorporated herein by reference). As stated above, the present invention also utilizes allogenic tumor cells. Allogenic tumor cells may be obtained from “model” patients, or alternatively may be derived from tissue culture cells.

The tumor cells of the invention may be live cells. Alternatively, the tumor cells and extracts of the present invention may be irradiated prior to use. Tumor cells or extracts are typically irradiated at about 2500 cGy to prevent cells from growing after injection.

In other embodiments of the invention, extracts or preparations containing less than all possible tumor cell components may be employed. Any extract or preparation may be employed, including preparations of isolated and purified individual components of tumor cells, or mixtures thereof. In general, extracts or preparations containing cell membranes, or membrane components, are generally preferred since it is expected that immune reactions directed to cell surface markers will generally be more effective. However, in certain circumstances, it may be desirable to use other preparations. For example, when it is desired to induce an immune reaction against a tumor cell component that is normally intracellular but is so over-expressed in tumor tissue that it can be expected to be presented with reasonable frequency on the cell surface in the context of MHC molecules. Alternatively or additionally, it may be desirable to direct an immune reaction against secreted agents expected to remain within the vicinity of the tumor.

Where purified tumor cell components are utilized, they may be prepared by any available means. For example, components may be purified directly from tumor cells, or may be isolated from other cells, even unrelated cells, that have been engineered (for example, by recombinant DNA technology) to express them. For example, peptides may be eluted from the surface of an autologous or allogenic tumor-cell (Yu et la. Cancer Res. 61(3):842-847 (2001)). Alternatively, components may be synthesized.

Generally, where any tumor cell component preparation other than intact tumor cells is employed, it is preferred that the preparation include one or more tumor-associated components. Tumor-specific components are particularly preferred.

Also, particularly preferred tumor cell components are those that are susceptible to haptenization by the sensitizing agent. Proteins or polypeptides are particularly preferred tumor cell components. Certain preferred embodiments of the invention utilize peptides corresponding to a portion of a tumor-associated or tumor-specific component (discussed further below).

Coupling Haptens and Tumor Cell Components

Those of ordinary skill in the art will appreciate that the strategy employed to associate inventive sensitizing agents or haptens may vary depending on the particular sensitizing agent/hapten and tumor cell component employed.

In general, sensitizing agent/hapten and tumor cell components are combined together under conditions and for a time sufficient for association to occur. In certain preferred embodiments of the invention, this association involves haptenization of tumor cell components by the sensitizing agent. In such embodiments, it is desirable to combine the tumor cell components with the sensitizing agent under conditions that allow covalent bond formation between the sensitizing agent/hapten and one or more tumor cell components. It will often, though not always, be preferred that the haptenized tumor cell components be at least tumor-associated, if not tumor specific. Furthermore, it will often be preferred that the haptenized tumor cell components be ones that the host immune system can encounter in the context of an intact tumor. For example, dendritic cells may include the host cells that encounter the haptenized tumor cell components. Dendritic cells are antigen-presenting cells that play a major role in stimulating the host's immune system. One method of improving the antigen-presentation by dendritic cells is by recruiting them to the site of the haptenized tumor cell components so that they contact the haptenized tumor cell component and display the haptenized tumor cell components on the cell surface. A method by which dendritic cells can be recruited is by administering to the site of the haptenized tumor cell components an injection of granulocyte-macrophage colony-stimulating factor (Nasi et al. Cytokines Cell. Mol. Ther. 5(3): 139-144, 1999).

In certain embodiments of the invention, sensitizing agent/hapten and tumor cell components are simply mixed together and allowed to associate before being administered to a patient. Generally, it is desirable to control reaction conditions so that haptenization is allowed or promoted, either prior to or after administration of the sensitizing agent/hapten and tumor cell component to a patient.

In other embodiments of the invention, one or more modification, isolation, purification or other steps is performed between the mixing of sensitizing agent/hapten and tumor cell component and the delivery of the combination to a patient. For example, one or more steps may be performed to ensure or promote haptenization. Alternatively or additionally, one or more purification steps may be performed, preferably to isolate sensitizing agent/tumor cell component complexes. For example, in some embodiments of the invention, intact tumor cells are mixed with sensitizing agent so that the sensitizing agent haptenizes certain proteins or other compounds on the surface of the tumor cells. The tumor cells are subsequently disrupted and hapten-cell component conjugates are isolated for administration to a patient.

Alternatively or additionally, one or more “presentation” steps, in which the manner by which the tumor cell component and sensitizing agent are presented to the patient is modified, may be performed. In one particularly preferred embodiment of the invention, haptenized tumor cell components are presented to a patient in the context of an antigen presenting cell (APC). For example, a variety of techniques have been developed for pulsing isolated APC, e.g., dendritic cells, with a particular antigen to promote presentation of the antigen in the context of MHC on the cell surface; cells can be pulsed with other cells, purified components of cells, or with antigen (see, for example, Ferlazzo et al. Int. Immunol 12(12):1741-1747, 2000; Lu et al. Infect. Immun. 67(4):1763-1769, 1999; Tjoa et al. Immunol Lett 74(1):87-93 (2000); each of which are incorporated herein by reference). In certain versions of this embodiment of the invention, the isolated APC are dendritic cells isolated from the individual whose tumor(s) is/are to be treated. The dendritic cells are presented in vitro with a sensitizing agent/tumor cell component mixture and allowed to process the mixture so that haptenized tumor cell peptides are displayed on the dendritic cell surface. The dendritic cells loaded with the haptenized tumor cell component may then be administered in vivo.

Encapsulation

In one particularly preferred embodiment of the invention, the inventive sensitizing agent/tumor cell component mixture is provided in association with an encapsulation device. Preferred encapsulation devices are biocompatible and are stable inside the body so that sensitizing agent/tumor cell component mixture is not released until after the encapsulation device is taken up into antigen-presenting cells. For example, preferred systems of encapsulation are stable at physiological pH and degrade at acidic pH levels comparable to those found in the endosomes of APCs. Preferably, the encapsulation device is taken up into antigen-presenting cells via endocytosis in clathrin-coated pits. Particularly preferred encapsulation compositions included but are not limited to ones containing liposomes, polylactide-co-glycolide (PLGA) (Gupta et al. Dev. Biol. Stand. 92:63-78, 1998), chitosan, synthetic biodegradable polymers, environmentally responsive hydrogels, and gelatin PLGA nanoparticles.

To elaborate on but one example of an encapsulation device, liposomes are well known to have the ability to channel protein and peptide antigens into the MHC class II pathway of phagocytic antigen-presenting cells, e.g., macrophages, to enhance the induction of antibodies and antigen-specific T cell proliferative responses. In addition, liposomes also deliver protein and peptide antigens, such as the haptenized tumor cell components of the present invention, into the MHC class I pathway to induce cytotoxic T cell responses. (See, for example, Rao et al., Adv Drug Deliv. Rev. March 30 41(2):171-188, 2000; Monte et al. J. Immunol 142(5):1437-1443, 1989).

Encapsulating devices may vary in size, shape molecular weight and/or ratio of materials that make up the encapsulation device (e.g., ratio of polymers). The inventive haptenized tumor cell components may be encapsulated in combination with one or more adjuvants, targeting entities, or other agents including, for example, pharmaceutical carriers, diluents, excipients, oils, etc. Alternatively or additionally the encapsulation device itself may be associated with a targeting entity and/or an adjuvant.

Adjuvants

A large number of adjuvant compounds is known; a useful compendium of many such compounds is prepared by the National Institutes of Health and can be found on the world wide web (see Allison Dev. Biol. Stand. 92:3-11, 1998; Unkeless et al. Annu. Rev. Immunol. 6:251-281, 1998; and Phillips et al. Vaccine 10:151-158,1992, each of which is incorporated herein by reference) and/or cytokines (e.g., IL-2, IL-7 etc.),

In some embodiments of the invention, the adjuvant is associated (covalently or non-covalently, directly or indirectly) with the haptenized tumor cell component so that adjuvant and haptenized tumor cell component(s) are delivered substantially simultaneously to the individual, optionally in the context of a single composition. In other embodiments, the adjuvant is provided separately. Separate adjuvant may be administered prior to, simultaneously with, or subsequent to haptenized tumor cell component administration. In certain preferred embodiments of the invention, a separate adjuvant composition is provided that can be utilized with multiple different haptenized tumor cell component compositions.

Where adjuvant and haptenized tumor cell components are provided together, any association sufficient to achieve the desired immunomodulatory effects may be employed. Those of ordinary skill in the art will appreciate that covalent associations will sometimes be preferred. For example, where adjuvant and tumor cell component are both polypeptides, a fusion polypeptide may be employed. To give another example, CpG-containing nucleotides may readily be covalently linked with tumor cell components. Those of ordinary skill in the art will be aware of other potential desirable covalent linkages.

Targeting

Inventive haptenized tumor cell components may desirably be associated with a targeting entity that will ensure their delivery to a particular desired location. In preferred embodiments of the invention, haptenized tumor cell components are targeted for uptake by antigen presenting cells. For example, haptenized tumor cell components may be targeted to dendritic cells or macrophages via association with a ligand that interacts with an uptake receptor such as the mannose receptor or an Fc receptor. Macrophages are any large amoeboid mononuclear cell, regardless or origin, such as not limited to histocytes and monocytes, which phagocytose, i.e., engulf and destroy other cells, dead tissue, degenerated cells, and the like. Macrophages are antigen presenting cells that present antigens, including tumor antigens, to cells including T cells Dendritic cells are also antigen presenting cells and appear to be closely related to macrophages, however, dendritic cells are more efficient antigen presenting cells then macrophages. They are potent stimulators of T cells and may be isolated from a variety of body organs and tissues including, but not limited to blood-skin (where dendritic cells are referred to as Langerhans cells), and lymphoid tissue. Method of isolating macrophages and dendritic cells are described, for example, WO 96/40173, supra.

Haptenized tumor cell components may also be targeted to other APCs via association with a ligand that interacts with the complement receptor. For example, haptenized tumor cell components can be specifically directed to dendritic cells through association with a ligand for DEC205, a mannose-like receptor that is specific for these cells.

Alternatively or additionally, haptenized tumor cell components could be targeted to particular vesicles within APCs. Those of ordinary skill in the art will appreciate that any targeting strategy should allow for proper uptake and processing of haptenized tumor cell components by the APCs.

Haptenized tumor cell components of the present invention can be targeted by association with an Ig molecule, or portion thereof. Ig molecules are comprised of four polypeptide chains, two identical “heavy” chains and two identical “light” chains. Each chain contains an amino-terminal variable region, and a carboxy-terminal constant region. The four variable regions together comprise the “variable domain” of the antibody; the constant regions comprise the “constant domain.” The chains associate with one another in a Y-structure in which each short Y arm is formed by interaction of an entire light chain with the variable region and part of the constant region of one heavy chain, and the Y stem is formed by interaction of the two heavy chain constant regions with one another. The heavy chain constant regions determine the class of the antibody molecule, and mediate the molecule's interactions with class-specific receptors on certain target cells; the variable regions determine the molecule's specificity and affinity for a particular antigen.

Class-specific antibody receptors, with which the heavy chain constant regions interact, are found on a variety of different cell types and are particularly concentrated on professional antigen presenting cells (pAPCs), including dendritic cells. According to the present invention, inventive compositions may be targeted for delivery to pAPCs through association with an Ig constant domain. In one embodiment, an Ig molecule is isolated whose variable domain displays specific affinity for the haptenized tumor cell components to be delivered, and the haptenized tumor cell components are delivered in association with the Ig molecule. The Ig may be of any class for which there is an Ig receptor, but in certain preferred embodiments, is an IgG. Also, it is not required that the entire Ig be utilized; any piece including a sufficient portion of the Ig heavy chain constant domain is sufficient. Thus, Fe fragments and single-chain antibodies may be employed in the practice of the present invention.

In one embodiment of the invention, a haptenized tumor cell component (e.g., a haptenized tumor cell antigen) is prepared as a fusion molecule with at least an Ig heavy chain constant region (e.g., with an Fe fragment), so that a single polypeptide chain, containing both haptenized tumor cell component and Ig heavy chain constant region components, is delivered to the individual (or system). This embodiment allows increased flexibility of haptenized tumor cell component selection because the length and character of the tumor cell component is not constrained by the binding requirements of the Ig variable domain cleft. In particularly preferred versions of this embodiment, the tumor cell component and the Fe portion of the fusion molecule are separated from one another by a severable linker that becomes cleaved when the fusion molecule is taken up into the pAPC. A wide variety of such linkers is known in the art. Fe fragments may be prepared by any available technique including, for example, recombinant expression (which may include expression of a fusion protein) proteolytic or chemical cleavage of Ig molecules (e.g., with papain), chemical synthesis, etc.

Pharmaceutical Compositions

The present invention further provides pharmaceutical compositions that comprise the inventive haptenized tumor cell component(s), as described in detail above, and a pharmaceutically acceptable carrier. It will be appreciated that the inventive pharmaceutical compositions encompasses the use of any haptenized tumor cell component that induces, stimulates, supports, or enhances an immune reaction in a host. It will also be appreciated that certain of the haptenized tumor cell components of the present invention can exist in free form for treatment.

As described above, the pharmaceutical compositions of the present invention additionally comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Fifteenth Edition, E. W. Martin (Mack Publishing Co., Easton, Pa., 1975) discloses various carriers used in formulating pharmaceutical compositions and known techniques for the preparation thereof. Except insofar as any conventional carrier medium is incompatible with the anti-cancer compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening agents, flavoring agents, and perfuming agents, preservatives, and antioxidants can also be present in the composition, according to the judgment of the formulator.

Administration of Pharmaceutical Compositions

The compositions of the present invention may be employed to induce, stimulate, support, or enhance an immune reaction in any animal. Preferably, the animal is a domesticated mammal (e.g., a dog, cat, horse, sheep, pig, goat, cow, bird, or lower mammal etc); more preferably, the mammal is a human. In a related embodiment, the compositions may be used to treat or prevent cancer. Individuals to whom the inventive compositions may be administered include any animal or human having one or more metastatic or primary tumors. For example, any individual who suffers from cancer or who is at risk of developing cancer may be treated. It will be appreciated that an individual can be considered at risk for developing cancer without having been diagnosed with any symptoms of cancer. For example, if the individual has a particular genetic marker identified as being associated with increased risk for developing cancer, that individual will be considered at risk for developing cancer. Similarly, if members of an individual's family have been diagnosed with cancer, the individual may be considered to be at risk for developing cancer. In addition, an individual who has been identified to have preneoplastic cells or polyps (e.g., polyps of the colon) may be an individual at risk for developing cancer.

The compositions of the present invention may be formulated for delivery by any route. Preferably, the compositions are formulated for injection. Alternatively, the compositions are formulated for ingestion, or inhalation. The compositions are administered in such amounts and for such time as is necessary to achieve the desired result. As described above, in certain embodiments of the present invention a “therapeutically effective amount” of an inventive sensitizing agent/tumor cell component mixture or pharmaceutical composition is that amount effective for attenuating, stimulating, supporting, or enhancing an immune reaction in any animal. The inventive mixtures and compositions, according to the method of the present invention, may be administered using any amount and any route of administration effective for stimulating, supporting, or enhancing an immune reaction in any animal. Thus, the “amount effective to attenuate, stimulate, support, or enhance an immune reaction in any animal”, as used herein, refers to a nontoxic but sufficient amount of the inventive sensitizing agent/tumor cell component mixture or pharmaceutical composition to stimulate, support, or enhance an immune reaction in any animal. As but one example, the “therapeutically effective amount” can be an amount to treat or prevent caner. Alternatively, the inventive sensitizing agent/tumor cell component mixture of the invention can be used to attenuate an immune reaction.

The exact amount required will vary from subject to subject, depending on the species, age, and general condition of the subject, the stage of the cancer, the particular sensitizing agent/tumor cell component mixture, its mode of administration, and the like. The sensitizing agent/tumor cell component mixtures of the invention are preferably formulated in dosage unit form for ease of administration and uniformity of dosage. The expression “dosage unit form” as used herein refers to a physically discrete unit of sensitizing agent/tumor cell component mixture appropriate for the patient to be treated. It will be understood, however, that the total daily usage of the compounds and compositions of the present invention will be decided by the attending physician within the scope of sound medical judgment. The specific therapeutically effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Particularly preferred preparations are injectable (e.g., intravenous) preparations. Injection of the pharmaceutical compositions of the invention is to a site in the body that will stimulate an immune response in an individual, e.g., a systemic immune response may be induced by injection into the arm. Alternatively, injection of the pharmaceutical compositions of the invention to a site in the body where it is desired that an immune reaction be stimulated, supported, or enhanced may be particularly effective. Injection of the inventive pharmaceutical composition directly into a tumor, for example, may result in an immune response at the site of the tumor, e.g., to reduce the size of the tumor. In an alternative embodiment, injection of the inventive pharmaceutical composition directly into the thymus or into one or more lymph nodes, where there are high concentrations of T cells, may maximally expose the T cells of the patient or organism to the inventive sensitizing agent/tumor cell component mixture.

For example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

In other embodiments, after formulation with an appropriate pharmaceutically acceptable carrier in a desired dosage, the pharmaceutical compositions of this invention can be administered to humans and other animals orally, rectally, parenterally, intracisternally, intravaginally, intraperitoneally, topically (as by powders, ointments, or drops), bucally, as an oral or nasal spray, or the like, depending on the location or severity of the cancer being treated. In certain embodiments, the compounds of the invention may be administered orally or parenterally at dosage levels of about 0.1 mg/kg to about 50 mg/kg and preferably from about 2 mg/kg to about 25 mg/kg, of patient body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

As discussed above, the compounds of the present invention are useful as anti-cancer agents, and thus may be useful in the treatment or prevention of cancers of any type.

It will also be appreciated that the compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another anti-cancer agent), or they may achieve different effects.

For example, the cancer cells or membranes may be co-administered with a peptide, any of which may be haptenized. In addition, the haptenized cancer cells, extracts, or peptides of the invention may be co-administered with other compounds, including, but not limited to cytokines, such as interleukin-2, interleukin-4, gamma interferon, interleukin-12, GM-CSF etc.

Other anti-cancer agents may be administered prior to, concurrently with, or after administration of an inventive pharmaceutical compositions of the present invention. For example, the inventive sensitizing agent/tumor cell component composition may be administered alone or in combination with one or more additional factors, such as adjuvants (e.g., BCG, alum, Freund's, DETOX, QS-21, CpGs, Listeria, etc.); anti-angiogenesis factors, etc., in order to enhance the immune response to the inventive composition, and/or the effectiveness of the overall anti-cancer treatment.

Also, it will be understood that the inventive techniques and reagents may be administered instead of or in addition to traditional anti-cancer therapies. For example, in some embodiments of the invention, tumors are first treated with radiation, chemotherapy surgery, antibodies, oligonucleotide sequences, and/or antisense oligonucleotide sequences prior to or concurrently with administration of the inventive compositions. For a more comprehensive discussion regarding cancer, symptoms and treatment, see The Merck Manual, Seventeenth Ed. 1999, the entire contents of which are hereby incorporated by reference.

In still another aspect, the present invention also provides a pharmaceutical pack or kit comprising one or more containers filled with one or more of the ingredients of the pharmaceutical compositions of the invention, and in certain embodiments, includes an additional approved therapeutic agent for use as a combination therapy. Optionally associated with such container(s) can be a notice in the form prescribed by a governmental agency regulating the manufacture, use or sale of pharmaceutical products, which notice reflects approval by the agency of manufacture, use, or sale for human administration.

### Equivalents

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art. The following examples contain important additional information, exemplification and guidance which can be adapted to the practice of this invention in its various embodiments and the equivalents thereof.

### EXAMPLES

### Example 1

### Preparation and Administration of Sensitizing Agent/Tumor Cell Composition

In this Example, tumor cells are haptenized with a sensitizing agent.

Crude Poison Ivy Extract/Urushiols

Extract from the leaves of poison ivy (Toxicodendron radicans or Rhus) is prepared as described in Smith et al., Dermatology 195(2):145, 1997; Vidmar et al., Am. J. Contact Dermat. 10(4):190, 1999; or Kalish et al., J. Invest. Dermatol. 92(1):46, 1989, each of which is incorporated herein by reference.

Rhus extract is made by extracting 18 g Toxicodedron radicans (poison ivy) leaves with 15 ml of demethylsulfoxide (DMSO) for 15 h at 4° C. The material is then filtered through a 0.20 μm filter, aliquotted, and stored at −70° C. Potency is determined by the ability to induce proliferation of PBM from rhus sensitive donors.

Alternatively, pure urushiol is prepared from Toxicodendron radicans or synthetic pentadecylcatachol (PDC) according to (Kalish et al., supra; Byck et al., supra; Kurtz et al., supra, supra; Lepoittevin et al., supra; ElSohly et al., supra; Stampf et al., supra; Dunn et al., supra; or Kalish et al. J. Clin. Invest. 93:2039, 1994, incorporated herein by reference). Analysis of the purified urushiol by gas chromatography typically shows no saturated side chains and a minimum of 95% catechol.

Preparation of Haptenized Tumor Cells

Preparation of haptenized non-tumor cells or tumor cells, (e.g., cell haptenize with the poison ivy extract or urushiols), requires first processing the tumor and then manufacturing the vaccine composition. Autologous or allogenic tumor tissue is obtained by surgical removal from a patient (e.g., from regional lymph node metastases or from subcutaneous or visceral metastases and processed into a single cell suspension using collagenase and DNAse. The aliquots of the tumor cells are pipeted into cryovials with DMSO as a cryopreservative, frozen in a controlled-rate freezer, and stored in liquid nitrogen. A vaccine composition is prepared on the day that a patient is to receive it. On that day, a vial of cells is thawed and washed. The cells are then irradiated to 2500 cGy. Cell may be modified with the poison ivy extract or urushiol beofre or after cryopreservation. This is achieved by incubating the tumor cells at room temperature for at least 4 h with the extract or urushiol.

Conjugation of Urushiols to Tumor Cells

The urushiol or extract proteins are conjugated to the tumor cells using standard techniques. Urushiols are oxidized to a more reactive quinone species that is capable of binding to proteins and other factors, thus conjugating the urushiol to a protein that is a tumor cell component. In general, the quinone derivatives and other derivatives of urushiol are capable of forming associations with proteins or other components of tumor cells.

Encapsulation of Tumor Cell/Crude Poison Ivy Extract Mixture

Optionally, the hapten modified tumor cells are encapsulated, e.g., by liposomes (Rao et al., Adv Drug Deliv. Rev. March 30 41(2):171, 2000; Monte et al. J Immunol 142(5):1437, (1989); Gregiruabdus et al. Methods 19:156, (1999), each of which is incorporated herein by reference), for delivery to the patient.

The following procedure has been adapted from Gregoriadis et al., supra, which is characterized by its mildness and involves a dehydration-rehydration procedure for the entrapment of liable materials.

Solutions required: (1) PC or DSPC, CHOL, PG, and TO (4:4:2:1 molar ratio, 9 □mol total lipid) in 1.0 ml CHCl₃. (2) Lipids as in solution 1 dissolved in 0.5 ml diethyl ether. (3) 0.15 M sucrose in H₂O. (4) 0.2 M sucrose in H₂O. (5) 5% glucose in H₂O. (6) 0.1 M sodium phosphate buffer supplemented with 0.9% NaCl, pH 7 (PBS). (7) Discontinuous sucrose gradient prepared by the use of two solutions containing 59.7 g and 117.0 g of sucrose, respectively, per 100 ml H₂O in swing bucket centrifuge tubes.

One milliliter of solution 3 is mixed by vortexing for 45 seconds with solution 1. The resulting water-in-chloroform emulsion is mixed by vortexing for 15 seconds with solution 2 and 2.5 ml solution 4. The water-in-oil emulsion formed is placed in a 250-ml conical flask and the organic solvents are evaporated by flushing N₂ at 37° C. while the sample is gently agitated in a shaking incubator. This leads to the generation of (sucrose-containing) giant liposomes. The giant liposomes are washed by centrifugation over solution 5 in a bench centrifuge at 600 g for 5 minutes. The liposomal pellet is then resuspended in 1 ml PBS. The resuspended pellet of giant liposomes is mixed with 1 ml of a suspension of hapten modified tumor cells and freeze-dried overnight in a vacuum (<0.1 Torr) in a Hetosicc freeze-dryer.

The freeze-dried material is rehydrated initially by the addition of 0.1 ml H₂O at 20° C. The suspension is then swirled vigorously and allowed to stand at TC for 30 minutes. The process is repeated after the successive addition of 0.1 ml PBS and 0.8 ml PBS 30 minutes later (1 ml total suspension volume).

The entrapped particulate material is separated from nonentrapped material (hapten modified tumor cells) by sucrose gradient centrifugation.

The suspension (1 ml) containing entrapped and nonentrapped material is placed on top of the sucrose gradient (solution 7) and centrifuged for 0.5 h at 90,000 g. Following centrifugation, 1 ml fractions are pipetted out from the top of the gradient and assayed for content. It is convenient to use radiolabeled material to follow the movement of fractions of the hapten modified tumor cells. The fractions containing the material are identified and recovered.

Fractions containing the hapten modified tumor cells are pooled and dialyzed exhaustively against PBS until all sucrose has been eliminated. The dialyzed material is centrifuged as in step D and the liposomal pellet resuspended in 1 ml PVS for further use.

Administration of Tumor Cell/Crude Poison Ivy Extract Mixture

Encapsulated or unencapsulated haptenized tumor cells are administered to a patient by following any number of variations of routine protocols. Examples of methods of sensitization and vaccination are provided in WO 96/40173, supra.

Patients are first sensitized on two consecutive days to the urushiol or extract preparation by topical application of a dilute solution of urushiol or extract (e.g., a 1:10 dilution). Cyclophosphamide, 300 mg/M² is administered intravenously three days prior to sensitization. Two weeks later, patients are again given cyclophosphamide followed three days later by administration of the haptenized tumor cells. Administration of the cyclophosphamide/haptenized tumor cell vaccine is repeated every 28 days. Alternatively, the vaccine composition is administered weekly for up to six weeks with cyclophosphamide given three days prior to administration. The vaccine composition consists of 10-25×10⁶ cryopreserved, autologous, irradiated, haptenized tumor cells prepared as described herein. BCG, an excellent immunologic adjuvant, is mixed with the vaccine of haptenized tumor cells just prior to administration. (Other protocols for vaccine preparation and administration are known in the art and described, for example, in WO 96/40173, incorporated herein by reference).

The mixture of haptenized tumor cells and BCG is injected intradermally into three contiguous sites, usually on the upper dorsal arm, but excluding arms ipsilateral to a lymph node dissection. Multiple injections are given by one of the dosage schedules, but all injections are given into the same extremity.

### Example 2

### Pulsing Antigen Presenting Cells with Urushiol Conjugated Tumor Antigen

In this Example, antigen presenting cells are pulsed with urushiol-conjugated tumor antigen.

Conjugation of Tumor Antigen with Urushiol

Urushiols are obtained as described in Example 1, above. The urushiol is conjugated to a known tumor antigen. The tumor antigen is a preparation of peptides eluted from the surface of autologous tumor cells that are harvested as described above in Example 1 (Yu et al., supra). Alternatively, the tumor antigen is one of a variety of tumor antigens that induce, stimulate, support, or enhance an immune reaction in a host against the tumor, which have been identified and used in cancer vaccines (see Table 1).

TABLE 1
Tumor Antigens

| Antigen | Application |
|---|---|
| p53 | potential target antigens for a broad- |
| Her-2/neu | spectrum, cytotoxic T lymphocyte-based |
| hdm2 | immunotherapy of various cancers and |
| CD1 | hematologic malignancies |
| | Voss et al. Bone Marrow Transplant 26 |
| | Suppl, 2000 |
| CA15-3 | breast cancer antigen |
| | Jiang et al. Cancer Biother. Radiophar. |
| | 15(5): 495, 2000 |
| CEA | carcinoembryonic antigen |
| | Jiang et al., supra |
| CA125 | ovarian cancer antigen |
| | Jiang et al. supra |
| PSA | prostate cancer antigen |
| | Meidenbauer et al. Prostate 43(2): 88, 2000 |
| PAP | prostate cancer antigen |
| | Tjoa et al. Immunol Lett 74(1): 87, 2000 |
| PSMA | prostate cancer antigen |
| | Tjoa et al. supra |
| MUC1 | broad-based tumor antigen |
| | von Mensdorff-Pouilly et al. Int. J. Biol. |
| | Markers 15(4): 343, 2000 |
| MAGE-3 | melanoma antigen and bladder cancer |
| | antigen |
| | Nishiyama et al. Clin. Cancer Res. 7(1): 23, |
| | 2001 |
| gp100 | melanoma antigen |
| | Mendiratta et al. Cancer Res. 61(3): 859, |
| | 2001 |
| g2009-2M | synthetic peptide vaccine derived from the |
| | sequence of gp100 melanoma-associated |
| | antigen |
| | Fritsch et al. J. Immunother. 23(5): 557, |
| | 2000 |
| Melan A/MART | melanoma antigen |
| | Reynolds et la. J. Immunol Methods 244(1-2): |
| | 59, 2000 |
| tyrosinase | melanoma antigen |
| | Reynolds et la. supra |
| TRP-2 | melanoma antigen |
| | Mendiratta et al., supra |
| Her-2/neu | breast cancer antigen |
| | Murray et al. Semin. Oncol. (6 Suppl. 11): |
| | 71, 2000 |
| gp96 | a heat shock protein-peptide, broad-based |
| | tumor antigen |
| | Janetzki et al. Int. J. Cancer 88(2): 232, |
| | 2000 |
| Ny-ESO-1 | cancer-testis antigen |
| | Jager et al. Proc. Natl. Acad Sci. U.S.A. |
| | 97(22): 12198, 2000 |

Conjugation of the tumor antigen and the urushiol is accomplished as described in Example 1.

Pulsing Antigen Presenting Cells with Haptenized Tumor Antigen

Highly purified monocytes are isolated as described by Ferlazzo et la. (J. Immunol. 12(12):1741, 2000) from peripheral blood mononuclear cells (PBMC). PBMC are plated at approximately 10⁶ cells/well in 24-well plates (Costar, Cambridge, Mass.) in RPMI 1640 (Euroclone, Milan, Italy) without serum at 37° C. in the presence of 5% CO₂. After 1 h, non-adherent cells are removed by extensive washing. To check the purity of the monocyte populations, adherent cells are detached from a single well by incubation in ice-cold PBS (Sigma, Milan, Italy) supplemented with 0.2% EDTA (Euroclone), then counted and analyzed for CD 14 surface expression using flow cytometry. Such monocytes may be pulsed as described below for dendritic cells.

Dendritic cells are isolated using fluorescence activated cell sorting (FACS) directly from blood by negative selection, since dendritic cells lack most of the markers typical of other leukocyte populations (e.g., CD3, CD14, CD15, CD16, CD19, which are markers for T cells, macrophage monocytes, granulocytes, natural killer, and B cells, respectively) see, Steinman et al., Annu. Rev. Immunol. 9:271, 1991, Thomas et la. J. Immunol. 150:821, 1993, each of which are incorporated herein by reference. Negative selection is carried out using a cocktail of the lineage-specific monoclonal antibodies.

Alternatively, dendritic cells are cultured from CD34 hematopoietic progenitor cells isolated from human umbilical cords and bone-marrow or CD 14+ monocytes isolated from peripheral blood (Ferlazzo et la. J. Immunol. 12(12):1741, 2000; Berhard et al. Cancer Res 55:1099, 1995; Romani et al. J. Immunol. Methods 196:137, 1996; Sllusto et la. J. Exp. Med. 179:1109, 1994; Tjoa et al. Prostate 27:63, 1995, each incorporated herein be reference). Cytokines such as granulocyte-macrophage colony-stimulating factor (GM-CSF), interleukin-4 (IL-4), and tumor necrosis factor alpha (TNF-α) are used to propagate the dendritic cells in vitro.

A population of dendritic cells capable of efficient antigen uptake can also be obtained by culturing monocytes for 7 days in RPMI 1640 medium containing 5% autologous serum, penicillin, and streptomycin (respectively 100 U/ml and 100 μg/ml), L-glutamine (2 mM) (Gibco, Grand Island, N.Y.), human recombinant granulocyte macrophage colony stimulating factor (GM-CSF, 25 ng/ml), and IL-4 (100 IU/ml; Euroclone). In some experiments CD cells are further treated with tumor necrosis factor TNF-α(25 ng/ml; Euroclone) and lipopolysaccharide (LPS, 100 ng/ml; Sigma) in order to induce a full dendritic cell maturation.

Dendritic cells are pulsed with the urushiol-conjugated antigen according to any of a variety of standard protocols, see Ferlazzo et al. Int. Immunol 12(12):1741, 2000; Ferlazzo et la. J. Immunol. 162:3597, 1999; or Lu et al. Infect. Immun. 67(4):1763, 1999; Tjoa et al. Immunol Lett 74(1):87, 2000; each of which are incorporated herein by reference.

For dendritic cell pulsing, 5×10⁵ dendritic cells are resuspended in 0.5 ml of medium containing the source of antigen (100 μg/ml protein 2.5×10⁶ irradiated cells of choice). Pulsing is carried out for 4 h at room temperature.

It will be appreciated that the pulsed dendritic cells may be optionally encapsulated, as described in Example 1, prior to administration to a cancer patient.

Administration of Pulsed Dendritic Cells

Dendritic cells are administered according to standard protocols (see Tjoa et al. supra, Murphy et al. Prostate 29:371, 1996, incorporated herein by reference). Pulsed dendritic cells are washed three times with 0.9% saline to eliminate trace medium, counted, and transferred in to a single-use 10-ml injectible 0.9% saline. Dendritic cells are infused over 30 minutes with a total volume of 100 ml 0.9% saline. Cancer patients receive four to six infusion cycles of the pulsed (autologous or allogenic) dendritic cells over six week intervals. With each infusion, patients receive a 7 day course of subcutaneous injection of GM-CSF as a systemic adjuvant.

Patients are monitored for cellular immune modulation to the appropriate antigen and regression of cancer, as described below. Methods of monitoring for regression of cancer vary depending on the particular type of cancer being treated. Those skilled in the art will recognize that such protocols may be varied according to the particular antigen selected or patient to whom the dendritic cells are being administered.

### Example 3

### Sensitizing Agent-Pulsed Antigen Presenting Cells

In this Example, dentritic cells are pulsed with sensitizing agent.

Crude poison ivy extract or purified urushiol is prepared and used as described above in Example 1. Dendritic cells are then pulsed with the poison ivy extract or urushiol preparation by incubation together for four hours at room temperature, as described in Example 1. The pulsed dendritic cells are administered to a cancer patient, as described in Example 2, and the immune response measured. It will be appreciated that the pulsed dendritic cells may be optionally encapsulated, as described in Example 1, prior to administration to a cancer patient or a patient in which an induced, stimulated, supported, or enhanced immune response is desired.

### Example 4

### Assays for Detecting Activity of Haptenized Compositions

In this Example, a variety of assays are described that are used to assess an immune reaction in a host. Any one or more of these assays may be used to test the patients of Example 1, 2 or 3.

Assays for Immune Reaction

The following is adapted from Sensi et al. J. Clin. Invest. 99:710, 1997; Berd et al. Annals New York Academy of Sciences 690:147, 1993; Berd et al. Seminars in Oncology 25(6);646, 1998; Berd et al. Cancer Res. 51:2731, 1991; Kalish et al. J. Clin. Invest. 93:2039, 1994; and Schepetkin Cancer Biotherapy & Radiopharmaceuticals 14(4):291, 1999.

Toxicity: Patients are examined to detect possible toxicity of the haptenized tumor cell composition. Some signs and symptoms of toxicity include the development of pruitic papules at the injection site that progress to pustules, sometimes with small ulcerations. The intensity of the reaction is ameliorated by progressively reducing the dose of BCG. Patients are also examined for low-grade fever and/or rash following administration. In addition, patients are monitored for autoimmune reactions such as arthritis or cutaneous vasculitis. Vertigo may also be a symptom of toxicity, as well as nausea and vomiting, often caused by low dose cyclophosphamide.

Inflammation: The patients are examined for the development of an immune response at the site of the tumor following administration. Such a response typically consists of marked erythema, warmth, and tenderness of the tumors and overlying skin. Inflammatory responses may vary in their time of appearance and may occur in one or more tumors. Inflammation is also measured by biopsing the tumor tissue to assess histologic changes (e.g., infiltration with lymphocytes, satellitosis of lymphocytes about the tumor cells, and local tumor necrosis. Immunohistochemical analysis of tumor biopsies are performed using an avidin-biotin peroxidase technique, as described by Lewis et al. (J. Invest. Dermatol. 93:672, 1989, incorporated herein by reference).

Flow Cytometry: Flow cytometric analysis of cell suspensions are prepared by enzymatic dissociation of excised tumors to enumerate tumor infiltrating lymphocytes. The cells are thawed and stained with monoclonal antibodies on ice for 30 minutes. The cells are then washed and analyzed with, e.g., a Coulter EPICS C flow cytometer (Coulter Electronics, Hialeah, Fla.). Lymphocytes are identified by antibody BH12 (American Type Culture Collection) to the T-200 common leukocyte antigen and are analyzed by setting appropriate bit map gates on a plot of forward versus 90-degree light scatter. The panel of monoclonal antibodies used for flow cytometry (and immunohistochemistry) includes Leu-1 (CD5), Leu-2 (CD8), Leu-3 (CD4), Leu-11 (CD16), NKH-1 (CD56), HLA-DR (L243), Leu-M1 (CD15), tac (Interleukin 2 receptor, CD25), and ICAM-1 (CD54), as well as any other monoclonal antibody that can identify cell-type recognized by one skilled in the art. Control samples for flow cytometric analysis include cell suspensions made from tumors excised without prior immunotherapy. Results indicate the presence or absence of infiltration of tumor tissue with, e.g., CD8+ and other immune cells.

Cytokine Production: The production of cytokines by lymphocytes infiltrating the tumor is studied by analyzing the tissues by a standard reverse-transcriptase polymerase chain reaction (RT-PCR) technique (Lattime et al. Cancer Immunol. Immunother. 41:151, 1995, incorporated herein by reference). Briefly, mRNA is harvested from tumor tissue using any standard protocol and subject to a RT-PCR (see also, for example, Molecular Cloning: A Laboratory Manual, 2nd Ed., ed. by Sambrook, Fritsch, and Maniatis (Cold Spring Harbor Laboratory Press: 1989); Methods in Enzymology (Academic Press, Inc., N.Y.); Ausubel et al. Current Protocols in Molecular Biology (John Wiley & Sons, Inc., New York, 1999), each of which is incorporated herein by reference).

Analysis of T-cell Receptor Structures: Whether the T cells infiltrating the tumor represent tumor-specific clones of T lymphocytes that have been expanded by the vaccine composition is also determined by performing a molecular analysis of the T-cell receptor (TCR) structures found within the inflamed tumors (Sensi et al. J. Clin. Invest. 99:710, 1997, incorporated herein by reference). Following administration of the vaccine composition, it is determined whether certain TCR structures predominate in the T lymphocytes extracted from the tumors compared with the heterogeneous patterns observed in prevaccine tumors and in peripheral blood.

Semiquantitave PCR is used as describe by Salvi et la. Cancer Res 55:3374, 1995. Total RNA is prepared using RNAzolB (Cinna/Biotecx, Rriendswood, Tex.), from thawed cryopreserved peripheral blood lymphocytes and enzymatically digested tumor samples. First-strand cDNA is synthesized with oligo-dT and reverse transcriptase (Superscript; GIBCO BRL, Gaithersberg, Md.). Serially diluted cDNA form all samples is amplified using TCR constant-region-specific primers. PCR products are electrophoresed on agarose gels, transferred to nylon membranes (Hybond N+; Amersham International, Little Chalfont, UK), hybridized with an internal ³²P-labeled TCR constant-region-specific oligonucleotide and scanned with a Phosphorlmager (No. 425;Molecular Dynamics, Sunnyvale, Calif.). Individual bands are digitized and integrated using the Imagequant software package provided by the manufacturer. The TCR□-chain variable gene repertoire analysis is then performed on the same amount of TCR □chain-specific cDNA template from PBL and tumor samples using a panel of described oligonucleotide primers that is determined by the type of cancer (e.g., see Genevee et al. Eru. J. Immunol. 22:1261, 1992). All experiments are performed at least twice. The levels of specific amplification are measured by densitometry and each band is expressed as a percentage of the sum of all signals detected in the repertoire analysis.

Clonality within selected gene TCR β-chain variable gene families is assessed by high resolution-polyacrylamide gel electrophoresis and single strand conformational polymorphism (SSCP) analysis as described by Gorski et al., J. Immunol. 152:5109, 1994) and Orita et al. Proc. Natl. Acad. Sci. U.S.A. 86:2766, 1989, incorporated herein by reference). TCR β-chain variable gene PCRs are performed by ³³P endlabeleing the TCR constant-region-specific antisense primer (0.08 μM). PCR products (5 μL) are heat denatured, separated both on 5% denaturing sequencing gel and on 6% nondenaturing polyacrylamine gels containing 10% glycerol and visualized by autoradiography.

Delayed Hypersensitivity: Delayed-type hypersensitivity (DTH) measurement is an accepted indicator of a cell-mediated immune response to an antigen. A positive DTH response indicates present or past exposure to an antigen, the best example being the purified protein derivative (PPD) response in tuberculosis. The DTH response to autologous tumor cell is used as an indicator of effectiveness of the inventive compositions in patients.

Patients are tested for cell mediated immunity to haptenized cells by skin testing with urushiol-conjugated mononuclear cells (MNC). Small numbers of irradiated tumor cells of the type to which the vaccine composition is targeted are injected intradermally into the ventral forearm. After 48 hours, DTH responses are apparent as urticaria-like reactions, which are quantitated as the mean diameter of the area of induration. One precautionary measure when evaluating responses using DTH response is to avoid scoring a non-specific response positive. For example, many human vaccines are prepared by dissociating tumors with the enzyme, collagenase, which is quite immunogenic. The test is thus performed in conjunction with autologous, irradiated tumor cells that have not been exposed to enzymes (e.g., cells that have been mechanically dissociated), or with another assay appropriate negative control. (See also, e.g., WO 96/40173).

Tumor reduction: Patients are monitored for a decrease in tumor size. Tumor size is assessed by any of a variety of methods, such as palpitation of the tumor, X-ray, CAT-scan, MRI, and the like. Patients in whom the tumors regress or can no longer be detected are followed up to determine the period of remission. Typically, patients are followed for at least five years.

### Example 5

### Evaluating Effects of Urushiol Conjugation on Tumor Cell Growth in Urushiol-Sensitized Mice

In this Example, we describe experiments that tested the effects of urushiol conjugation on tumor cell growth in urushiol-sensitized mice. Purified urushiol (ElSohly et al., J. Natural Prod. 45:532, 1982, incorporated herein by reference) was contacted with MethA fibrosarcoma cells (10 million cells in 1 ml mixed with 10 ug of urushiol in 100 ul DMSO for 1 hr at 37° C.). The cells were then washed.

Mice were sensitized with purified urushiol by exposing shaved skin to 50 ul urushiol (2 mg) per mouse. Two weeks later, the mice were tested for urushiol-specific contact hypersensitivity responses by exposing the right ear to 10 ul urushiol (10 mg) and measuring ear thickness.

Treated or untreated tumor cells were injected (1 million cells) into urushiol-sensitized mice two weeks after the mice were tested for contact hypersensitivity responses. Tumor diameter was measured daily with calipers.

### Other Embodiments

Those of ordinary skill in the art will appreciate that the foregoing represents certain preferred embodiments of the present invention and should not be construed to limit the spirit or scope of the invention as defined by the following claims.

1. A composition comprising:
a tumor cell component; and
a hapten linked to the tumor cell component to form a haptenized tumor cell component, which hapten is characterized in that it maintains the ability to stimulate, support, or enhance an immune reaction in a host when linked to the tumor cell component.

2. The composition of claim 1 wherein the hapten comprises an entity selected from the group consisting of compounds that are naturally produced by poison ivy, poison oak, or poison sumac plants.

3. The composition of claim 1 wherein the hapten is a urushiol.

4. The composition of claim 1 wherein the tumor cell component comprises at least one intact cancer cell.

5. The composition of claim 1 wherein the tumor cell component comprises at least one tumor associated peptide.

6. The composition of claim 5 wherein the hapten is linked to the tumor-associated peptide as a hapten-peptide conjugate, which conjugate is present in an MHC cleft on a surface of the antigen presenting cell.

7. The composition of claim 1 further comprising an antigen presenting cell.

8. The composition of claim 7 wherein the antigen presenting cell is selected from the group consisting of dendritic cells, macrophages.

9. The composition of claim 7 wherein the antigen presenting cell is a dendritic cell.

10. A method of treating cancer comprising:
administering to a subject suffering from cancer a composition comprising an effective amount of a tumor cell component linked to a hapten to form a haptenized tumor cell component, which hapten is characterized in that it maintains the ability to stimulate, support, or enhance an immune reaction in a host when linked to the tumor cell component.

11. The method of claim 10 wherein the hapten comprises an entity selected from the group consisting of compounds that are naturally produced by poison ivy, poison oak, or poison sumac plants.

12. The method of claim 10 wherein the hapten is a urushiol.

13. The method of claim 10 wherein the tumor cell component comprises at least one intact tumor cell.

14. The method of claim 10 wherein the tumor cell component comprises at least one tumor associated peptide.

15. The method of claim 14 wherein the hapten is linked to the tumor-associated peptide as a hapten-peptide conjugate, which conjugate is present in an MHC cleft on a surface of the antigen presenting cell.

16. The method of claim 10 further comprising administering an antigen presenting cell in combination with the haptenized tumor cell component.

17. The method of claim 16 wherein the antigen presenting cell is selected from the group consisting of dendritic cells, macrophages.

18. The method of claim 16 wherein the antigen-presenting cell is a dendritic cell.

19. The method of claim 16 wherein the antigen presenting cell is loaded in vitro with a haptenized tumor cell component.

20. The method of claim 19 wherein the antigen presenting cell is administered in vivo.

21. The method of claim 10 further comprising administering the haptenized tumor cell component with an adjuvant.

22. The method of claim 10 wherein the haptenized tumor cell component is encapsulated.

23. The method of claim 10 wherein the haptenized tumor cell component is targeted to an antigen-presenting cell.

24. The method of claim 10 wherein the haptenized tumor cell component is administered with a chemotherapeutic agent.

25. The method of claim 10 wherein the haptenized tumor cell component is administered with an anti-angiogenesis factor.

26. A composition comprising an antigen presenting cell, wherein the antigen presenting cell presents a sensitizing agent selected from the group consisting of compounds that are naturally produced by poison ivy, poison oak, or poison sumac plants.

27. The composition of claim 26 wherein the antigen presenting cell is a dendritic cell.

28. The composition of claim 26 wherein the antigen presenting cell is a macrophage.

29. The composition of claim 26 wherein the sensitizing agent is a urushiol.

30. The composition of claim 26 wherein the sensitizing agent is an extract of poison ivy.

31. The method of claim 26 wherein the antigen presenting cell presents a haptenized tumor cell component.

32. The method of claim 31 wherein the haptenized tumor cell component is a haptentized tumor cell.

33. The method of claim 31 wherein the haptenized tumor cell component is a tumor associated peptide

34. The method of claim 31 wherein the antigen presenting cell is administered to a patient in vivo.

35. A method of stimulating an immune response in an individual, the method comprising:
administering to a subject suffering from cancer a composition comprising and effective amount of a antigen presenting cell, wherein the antigen presenting cell presents a sensitizing agent selected from the group consisting of compounds that are naturally produced by poison ivy, poison oak, or poison sumac plants.

36. The method of claim 35 wherein the hapten is a urushiol.

37. The method of claim 35 wherein the sensitizing agent is an extract of poison ivy.

38. The composition of claim 35 wherein the antigen presenting cell is a macrophage.

39. The composition of claim 35 wherein the antigen presenting cell is a dendritic cell.

40. The method of claim 35 wherein the sensitizing agent is present in an MHC cleft on a surface of the antigen presenting cell.

41. The method of claim 35 wherein the antigen presenting cell is administered with an adjuvant.

42. The method of claim 35 wherein the antigen presenting cell is encapsulated.

43. The method of claim 35 wherein the sensitizing agent is targeted to an antigen-presenting cell.

44. The method of claim 35 wherein the antigen presenting cell is administered with a chemotherapeutic agent.

45. The method of claim 35 wherein the antigen presenting cell is administered with an anti-angiogenesis factor.
